(19) United States
(12) Patent Application Publication
(10) Pub. No.: US 2023/0383257 A1
(43) Pub. Date: Nov. 30, 2023

(54) PRODUCTION OF MEGAKARYOCYTES AND PLATELETS IN A CO-CULTURE SYSTEM

(71) Applicant: BOARD OF REGENTS, THE UNIVERSITY OF TEXAS SYSTEM, Austin, TX (US)

(72) Inventors: Elizabeth SHPALL, Houston, TX (US); Katy REZVANI, Houston, TX (US); Bijender KUMAR, Houston, TX (US); Mayela MENDT, Houston, TX (US); Vahid AFSHAR-KHARGHAN, Houston, TX (US); Rafet BASAR, Houston, TX (US)

(21) Appl. No.: 18/248,193

(22) PCT Filed: Oct. 15, 2021
(86) PCT No.: PCT/US2021/071903

§ 371 Date: Apr. 6, 2023

Related U.S. Application Data
(60) Provisional application No. 63/092,024, filed on Oct. 15, 2020.

(51) Int. Cl. C12N 5/078; A61K 35/19

(52) U.S. Cl. CPC C12N 5/0644; A61K 35/19; C12N 2506/1369; C12N 2502/11; C12N 2501/727; C12N 2501/125; C12N 2501/145; C12N 2501/2306; C12N 2501/724

(57) ABSTRACT

Embodiments of the disclosure include systems, methods, and compositions for producing megakaryocytes and platelets for recipient individuals in need thereof. The megakaryocytes and platelets are produced following co-culture of MSCs and CD34+ cells in media comprising stem cell factor, thrombopoietin, and IL-6, and wherein at least the CD34+ cells have a knock-in of HLA-E at the beta-2-microglobulin genomic locus, in specific embodiments. In some cases, ROCK inhibitors are utilized.

This application claims priority to U.S. Provisional Patent Application Ser. No. 63/092,024, filed Oct. 15, 2020, which is incorporated by reference herein in its entirety.

### TECHNICAL FIELD

Embodiments of the disclosure concern at least the fields of cell biology, molecular biology, cell culture, and medicine.

### BACKGROUND

More than 2 million platelet units are transfused annually across the US hospitals to treat the thrombocytopenia patients [1, 2]. There has been a constant demand for apheresis-derived platelet products for patients receiving chemotherapy, undergoing surgery, or who have underlying thrombocytopenia of any cause, for example. The current COVID-19 endemic outbreak in US is also another major concern and has caused unexpected over-utilization, leading to a platelet shortage. Platelets have a very short storage life, and hospitals are dependent on the donors to replenish the transfusion unit supply [3]. To overcome dependency on donors, there is a need for development of donor-independent and readily available platelets, including to be used for transfusion units. The present disclosure satisfies this need.

### BRIEF SUMMARY

Embodiments of the present disclosure are directed to systems, methods, and compositions that facilitate production of megakaryocytes and platelets. In specific embodiments, megakaryocytes produced in methods of the disclosure generate the platelets. Particular embodiments encompass specific reagents, conditions, timings, and/or certain cell manipulations to produce desired cells. Embodiments of the disclosure include systems and methods in which a linear sequence of events and specific, intentional steps result in expansion and differentiation and collection for desired cells, including megakaryocytes and/or platelets. The system and methods disclosed herein utilize selected media having desired reagents and conditions that facilitate the expansion and differentiation of particular cells. In some embodiments, the system may utilize a process including a series of steps (or, in some cases, steps that may be occurring substantially at the same time for different non-synchronous populations of cells in the same system). In any event, the disclosure provides a universal measure to overcome platelet transfusion-related refractoriness.

In particular embodiments, the present disclosure concerns co-culture of at least two populations of cells that allows expansion and differentiation of a specific, desired population of cells. In specific embodiments, an initial or at least early step in the system and process includes co-culture of mesenchymal stem cells (MSCs) with CD34+ cells (including CD34+-enriched stem cells). The use of allogenic MSCs is advantageous for providing superior support for stem cell expansion and differentiation. In specific embodiments, the system and methods of the disclosure avoid use of artificial extracellular matrices to prevent the apoptosis of the stem cells in co-culture. In particular embodiments, the MSCs and CD34+ cells are derived from a particular source, such as cord blood. Any CD34+ cells may be selected by positive enrichment or negative selection, or both.

The co-culture of the two populations of cells may be subjected to media that comprises one or more particular reagents, including stem cell factor (SCF), thrombopoietin (TPO), and IL-6. In some embodiments, the MSCs and/or the CD34+ cells have been manipulated to express one or more heterologous genes and/or to inhibit expression of one or more endogenous genes. In specific embodiments, the MSCs and/or the CD34+ cells are so manipulated prior to initiation of the expansion process. In some cases, the MSCs and/or the CD34+ cells are manipulated to have reduced or completely inhibited expression of endogenous Rho associated coiled—coil containing protein kinase (ROCK) in the MSCs and/or the CD34+ cells. In specific cases, endogenous ROCK1 (also called ROCK I, ROKβ, Rho-kinase β, or p160ROCK) and/or ROCK2 (also known as ROCK II, ROKα, or Rho kinase) have reduced or completely inhibited expression in the MSCs and/or the CD34+ cells. In some embodiments, any step or point(s) in time in the process may employ one or more ROCK inhibitors in the media for the cell culture; in a specific case, one or more ROCK inhibitors are utilized following production of megakaryocytes, such as during platelet production and/or harvest.

In particular embodiments, the MSCs and/or the CD34+ cells are manipulated and/or exposed to conditions that allow platelets produced therefrom to have an enhanced efficacy upon delivery to an individual in need thereof, including an individual that is allogenic with respect to the original source of the respective MSCs and/or the CD34+ cells. In at least some cases, the MSCs and/or the CD34+ cells are manipulated such that platelets ultimately produced by their co-culture do not elicit a deleterious immune system reaction in the recipient individual. In at least some cases, the MSCs and/or the CD34+ cells (including from Cord blood) are manipulated such that platelets ultimately produced by their co-culture are HLA-I depleted-derived megakaryocyte and platelets. In specific embodiments, the MSCs and/or the CD34+ cells are manipulated such that platelets ultimately produced by their co-culture are not destroyed by T cells and/or NK cells in the recipient individual. In specific embodiments, the MSCs and/or the CD34+ cells are manipulated to have a knock-in of HLA-E. The HLA-E knock-in may be anywhere at the beta-2-microglobulin (B2M) locus of the respective MSCs and/or the CD34+ cells. The knock-in may reduce, including completely deplete, the expression of HLA-I, including B2M, by the MSCs and/or CD34+ cells. Any manipulation of MSCs and/or the CD34+ cells may or may not be CRISPR-Cas9 mediated.

In particular embodiments, the system and methods include expansion, differentiation, and platelet production all in media that has the same composition (and that may or may not be changed at particular timepoints) and/or all in the same vessel, although in alternative embodiments different vessels are utilized and/or different steps of the process utilize media having different composition. The system is GMP-grade compliant, in specific embodiments. The system and methods may be serum-free, including free of bovine serum albumin or any other lipid supplements, in at least some cases.

The present MSC/CD34+ stem cell co-culture system allows for significantly improved the expansion potential of megakaryocytes in the co-culture system, including in specific embodiments at least 10-, 20-, 30-, 40-, 50-, 60-, 70-, 80-, 90-, 100-, 125-, 150-, 175-, 200-, 225-, 250-, 275-, 300-, 325-, 350-, 375-, 400-, 425-, 450-, 475-, 500-, 600-, 700-, 800-, 900-, or 1000-fold or greater compared to starting cells.

Embodiments of the disclosure include methods of producing megakaryocytes in an ex vivo system, comprising the step of co-culturing mesenchymal stem cells (MSCs) with CD34+ cells in one or more vessels or substrates in the presence of media comprising an effective amount of agents, said agents comprising, consisting essentially of, or consisting of stem cell factor (SCF), thrombopoietin (TPO) and interleukin 6 (IL-6), under conditions to produce the megakaryocytes, wherein the CD34+ cells have been manipulated to comprise a knock-in of HLA class I histocompatibility antigen, alpha chain E (HLA-E) at the genomic locus of β2-microglobulin (β2M) in the CD34+ cells, thereby reducing or eliminating expression of the HLA class I gene product in the CD34+ cells. In some embodiments, the method further comprises the step of enhancing production of platelets from the megakaryocytes. At least the majority of the CD34+ cells and/or the MSCs may be derived from cord blood, bone marrow, and/or adipose tissue. The vessel may further comprise an effective amount of one or more inhibitors of Rho-associated coiled coil containing protein kinase (ROCK), such as Y27632, GSK269962, Azaindole 1, RKI-1447, GSK429286a, GSK180736a, fasudil, hydroxyfasudil, or a combination thereof, and the one or more ROCK inhibitors may inhibit ROCK1 and/or ROCK2.

In particular embodiments, the media comprises or does not comprise one or more particular components or have certain concentrations for certain components. For example, the media may lack serum. In some cases, the concentration of SCF is in the range of 25-50 ng/mL; the concentration of TPO is in the range of 50-100 ng/mL; and/or the concentration of IL-6 is in the range of 50-100 ng/mL. In specific embodiments, the concentration of SCF, TPO, and IL-6 are substantially the same, such as about 50 ng/mL. In specific cases, the media comprises an effective amount of IL-1B.

In specific embodiments, at least part of the method comprises agitation of the one or more vessels or substrates. The agitation may occur during a co-culture, including the co-culture between MSCs and CD34+ cells. The agitation may occur during the production and/or harvesting of platelets. The agitation may or may not occur at a desired angle, such as about 8-9°. The agitation may be sufficient to induce shear stress on the megakaryocytes.

In particular embodiments, the megakaryocytes are reused to produce additional platelets. In certain aspects, cells are obtained from the media to analyze them for expression of one or more megakaryocyte markers (such as CD42b, CD41a, CD61, or a combination thereof). The cells may be obtained from the media about 10-12 days from the beginning of the co-culture. The cells may be obtained from the media about 22-24 days from the beginning of the co-culture. In certain embodiments, platelets are obtained from the media, including obtained from the media multiple times, and a certain duration of time between obtaining the platelets may be desired, such as about 3 days. Following procurement of the platelets at different, they may be combined. In any case, the platelets may be analyzed, such as analyzed for aggregation.

In certain embodiments, the method comprises the step of subjecting the MSCs, CD34+ cells, and/or megakaryocytes to an effective amount of one or more means of fucosylation of the CD34+ cells, MSCs, and/or megakaryocytes. The means of fucosylation may comprise one or more fucosyl-transferase enzymes along with GDP fucose substrate. The media may comprise an effective amount of one or more fucosyl-transferase enzymes.

In certain embodiments, there is a method of producing platelets that avoid a host individual's deleterious immune reaction, comprising the steps of: (a) co-culturing mesenchymal stem cells (MSCs) with CD34+ cells in one or more vessels or substrates in the presence of media comprising an effective amount of agents, said agents comprising, consisting essentially of, or consisting of SCF, TPO and IL-6, under conditions to produce the megakaryocytes, wherein the CD34+ cells have been manipulated to comprise a knock-in of HLA-E at the genomic locus of B2M in the CD34+ cells, thereby reducing or eliminating expression of HLA-I and/or B2M in the CD34+ cells, thereby producing megakaryocytes; and (b) subjecting the megakaryocytes to suitable conditions to produce an effective amount of the platelets. In some cases, the suitable conditions of step (b) comprise an effective amount of one or more ROCK inhibitors in the media. The steps of (a) and (b) may or may not occur in the same vessel or substrate.

In particular embodiments an effective amount of any platelets encompassed herein are provided to an individual in need thereof. In some cases, the individual in need thereof has cancer; thrombocytopenia; bone marrow disease; blood disease; anemia; aplastic anemia; coronavirus infection; is receiving and/or will receive an organ or bone marrow transplant; has a traumatic injury; is an individual undergoing and/or that will undergo heart surgery; is a burn victim; or a combination thereof.

In certain embodiments, there is a method of treating an individual in need of platelets, comprising the step of administering to the individual an effective amount of platelets produced by any method encompassed herein, wherein the individual has cancer; thrombocytopenia; bone marrow disease; blood disease; anemia; aplastic anemia; coronavirus infection; is receiving and/or will receive an organ or bone marrow transplant; has a traumatic injury; is an individual undergoing and/or that will undergo heart surgery; is a burn victim; or a combination thereof.

In some embodiments, there is a system comprising, consisting of, or consisting essentially of an effective amount of the following: MSCs; CD34+ cells, or, optionally, CD34+ cells comprising a knock in of HLA-E at the B2M genomic locus; a vessel or substrate; media; SCF; TPO; IL-6; and, optionally one or more ROCK inhibitors; and, optionally, one or more fucosyl-transferase enzymes.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1B. Schematic representation of a co-culture system of the disclosure. In specific cases, there is a strategy for the production of mature megakaryocytes from cord blood (CB) CD34+ cells in a MSC co-culture system that can be followed on Day 23 (for example) by transfer to liquid culture for additional days, with harvesting of megakaryocyte (MK)/platelets every 2-3 days. (FIG. 1B) Schematic representation of a strategy for the production of beta2—microglobulin knockout (KO) CB CD34+ cells or early megakaryocyte progenitors using CRISPR-Cas9 system followed by differentiation protocols used in FIG. 1A to produce genetically edited beta2-microglobulin KO mature megakaryocytes and platelets.

FIGS. 2A-2E. Expansion and characterization of CB derived CD34+-derived megakaryocytes. (FIG. 2A) Fold change in number of mature megakaryocytes after 20 days expansion with and without MSCs, number above bar indicates p-value. (FIG. 2B) 5× AXIO VertA1 image analysis of CD34+ cells in CFU-Meg conditions after 12 days (FIG. 2C) 20× image showing proplatelets-like extensions marked by black triangles in Meg-CFU conditions. (FIG. 2D) Giemsa staining analysis of a mature megakaryocyte. (FIG. 2E) Flow cytometry histogram plots of the day 20 expanded megakaryocytes showing CD41a, CD42b and CD61 expression.

FIGS. 3A-3C. Rock inhibitor(Y27632) treatment increases megakaryocyte polyploidy. (FIG. 3A) Schematic representation of the strategy for the rock inhibitor treatment of mature megakaryocytes over 5 days. (FIG. 3B) Histogram plots showing propidium Iodide (PI) staining in fixed and permeabilized untreated or Y27632 treated megakaryocytes. (FIG. 3C) Percentage of polyploid (=>8N) megakaryocytes in untreated or Y27632 treated groups, number above bar indicates p-value.

FIGS. 4A-4E. Rock inhibitor treatment enhance platelets secretion and TRAP induced activation. (FIG. 4A) Schematic representation of the strategy for the in-vitro megakaryocytes culture with or without rock inhibitor(Y27632) for 48 hrs. (FIG. 4B) Number of platelets after 48 hrs, number above bar indicates p-value. (FIG. 4C) MKs cell size after Rock inhibitor treatment (FIG. 4D) CD62P expression in platelets after TRAP stimulation. (FIG. 4E) Number of CD62P positive activated platelets, number above bar indicates p-value.

FIGS. 5A-5D. CB derived platelets circulate in immunodeficient mice and ex-vivo expanded megakaryocytes can engraft in various niches and release circulating platelets in the NOD scid gamma mouse (NSG) mice, a model of immunodeficiency. In particular, engrafted CB megakaryocytes can release circulating platelets, and ex-vivo generated CB functional platelets are detected in blood (FIG. 5A) Schematic representation of the strategy for the megakaryocytes transplantation and engraftment analysis. (FIG. 5B) megakaryocyte engraftment at 2 months in different organs in NSG mice. (FIG. 5C) The contour plots analysis showing the transplanted CB megakaryocytes derived platelets chimerism in the peripheral blood of NSG mice at 4 weeks after transplantation. (FIG. 5D) Ex-vivo generated platelets chimerism in mice blood at 0, 1, 4 and 24 hrs. time points.

FIGS. 6A-6C. CRISPR-Cas9 genome editing of cells. FIGS. 6A-6B show a graphical representation of CRISPR-Cas9 genome editing technology to generate beta2-microglobulin knockout(KO) CB-derived CD34+ cells. The cord blood (CB) CD34+ cells undergo electroporation with Cas9 protein and either single guide RNA (sgRNA; FIG. 6A) or dual guide RNAs, trans-activating crRNA(tracrRNA) and crispr RNAs(crRNA) hybrids in (1:1 ratio, FIG. 6B) specific targeting for beta2—microglobulin (HLA-I genes). The Cas9 generates double stranded breaks(DSB) in the target gene and removes the genomic DNA fragments, making the cells devoid of functional HLA-I protein complex. The cells will eventually repair through non homologous end joining (NHEJ) repair pathway, which repairs the broken ends without the donor DNA and results in the deletion (indel) mutations in the edited cells. These CB B2M KO CD34+ cells are fully functional and can be expanded and differentiated into megakaryocytes and platelets. (FIG. 6C) Schematic flow cytometry-based histogram plots analysis representing the B2M mean fluorescence intensity (MFI) expression in control (Cas9 only, red (peaks toward the right)) and CRISPR-Cas9 edited (blue (peaks toward the left) CD34+ cells, confirming B2M KO in around 85% CD34+ cells.

FIGS. 7A-7G. FIG. 7A. Flow cytometry based histogram analysis of HECA-452 antibody mean fluorescence intensity (MFI) expression in control and fucosylated megakaryocyte-erythroid progenitors (MEPs, lineage-CD34+CD38+CD135-CD45RA− cells) suggesting increased fucosylation levels in fucosylated MEPs. (p<0.0001, FIG. 7B) Flow cytometry-based histogram analysis of HECA-452 antibody mean fluorescence intensity (MFI) expression in control and fucosylated HSCs(lineage-CD34+CD38−CD90+CD45RA− cells) suggesting increased fucosylation levels after exogenous fucosylated HSCs (p=0.0058, FIG. 7B). (FIG. 7C) HECA-452 antibody mean fluorescence intensity (MFI) histogram expression in control and fucosylated megakaryocytes (CD41a+CD42b+CD61+ cells). (FIGS. 7D-7E) Schematic representation of CB megakaryocytes injection into NSG mice, BM homing analysis and the percentage of homed megakaryocytes in control and fucosylated megakaryocytes group. (FIGS. 7F-7G) CB megakaryocyte injection strategy into sublethally irradiated(3Gy) NSG mice and day 7 blood circulating CB derived platelets percentages measurement in the control and fucosylated groups.

### DETAILED DESCRIPTION

The present disclosure concerns production of desired cells from co-culture of at least two populations of starting cells; the production includes expansion and differentiation steps, followed by harvesting of the desired cells. In particular embodiments, megakaryocytes are produced from a co-culture system that includes at least stem cells as one of the starting populations. In at least some cases, one or more of the initial populations in the co-culture are from cord blood, including human cord blood.

In specific embodiments, the production of megakaryocytes from the human cord blood (CB) hematopoietic stem cells provides benefits for transfusion medicine. The present disclosure concerns an original approach for the large-scale generation of megakaryocytes (MK) from CB using CB tissue-derived mesenchymal stem cells (MSCs) in a c-culture system. The expansion and differentiation protocols of CB-derived CD34+ cells with MSC co-cultures has been optimized in particular cases to utilize certain reagent(s), condition(s), timing(s), and so forth. In specific embodiments, at least the expansion and differentiation protocols occur in serum free conditions supplemented with exogenous SCF, TPO, IL-6 cytokines (in some cases, each at a concentration of 50 ng/mL). In specific embodiments, FLT3-L (10-25 ng/ml), IL-21 (50-150 ng/ml), IL-9 (40-100 ng/ml), and/or IL-11 (10-100 ng/ml) cytokines can also be added to further enhance the expansion and differentiation potential of megakaryocytes.

MSC co-culture for the disclosed systems is advantageous in persevering the long-term functions of the hematopoietic stem cells and differentiating megakaryocytes, as it recapitulates the bone marrow microenvironment, where all cells lie in close proximity. These CB-derived ex vivo expanded cells express mature megakaryocyte lineage specific markers and secrete functional platelets, for example exhibiting CD62P (P-selectin) expression after thrombin receptor-activating peptides (TRAP) stimulation. In certain embodiments, the system and methods are further optimized at the step of the megakaryocyte maturation, platelet secretion, and/or their activation profile upon use of one or more Rho associated coiled—coil containing protein kinase (ROCK) inhibitors (commercially available, in at least some cases). The inventors demonstrate herein that the CB-derived mature megakaryocytes and their secreted platelets are physiologically active, can home and engraft successfully in a xenogeneic NSG mouse model and maintain long-term donor platelet chimerism in vivo. These expanded megakaryocyte progenitors provide short term platelet support for individuals in need thereof, including at least thrombocytopenic patients.

The presently disclosed systems and methods address a major problem in transfusion medicine today at least with respect to patients with refractory thrombocytopenia because of sensitization with HLA antibodies [4, 5]. The patients do not respond to platelet transfusions, even single donor, and they often experience serious and fatal bleeding complications [6, 7]. Certain presently disclosed systems and methods provide strategies to overcome alloimmune antibody-induced rejection of transfused platelets by utilizing genetic engineering, such as clustered regularly interspaced short palindromic repeats/Cas9(CRISPR-Cas9)-induced ablation, to reduce or ablate the expression of HLA genes, including the HLA-I complex molecule β2-microglobulin gene, leading to non-recognition by a host immune system and thereby escape from transfusion-induced thrombocytopenia. This allows for the infusion of CB-derived megakaryocyte progenitors and platelets to evade the alloantibody-mediated destruction and allow them to survive and provide robust platelet support.

### I. Systems and Methods of Production of Megakaryocytes and Platelets

The present disclosure concerns systems and methods for producing megakaryocytes, from which platelets may be produced. In some embodiments, the systems and methods utilize a co-culture system to produce megakaryocytes, and in specific cases they concern production of large scale clinical grade mature megakaryocyte and platelet products for any suitable clinical purpose, including therapeutic or preventative. In cases wherein the cells used in the initial step(s) of the system and process are from an individual in need of platelets, the platelets produced by the system and methods may be utilized for the individual in an autologous manner. In cases wherein the cells used in the initial step(s) of the system and process are from an individual or individuals that are not the recipient of the platelets, the platelets produced by the system and methods may be utilized for allogeneic recipients; in such cases, the produced platelets may be used in an off-the-shelf manner. Platelets that are off-the-shelf may or may not be suitably stored prior to use.

The presently disclosed systems and methods may produce high numbers (for example, at least 10⁹, 10¹⁰, 10¹¹, and so forth, including in a specific case 1-7×10¹¹) of mature megakaryocytes and functional platelets, including cord blood derived mature megakaryocytes and functional platelets. These mature megakaryocytes consistently secrete active platelets and providing a controllable source of platelets from any source, including from HLA-mismatched cord blood sources, for example. In specific embodiments of the disclosure, the mature megakaryocytes retain the ability to consistently produce functional platelets because of an intentional selection of one or more specific reagents, one or more specific conditions, one or more specific timings, and/or one or more specific certain cell manipulations. In specific embodiments, without such one or more specific reagents, one or more specific conditions, one or more specific timings, and/or one or more specific certain cell manipulations, the desired activity and/or numbers of megakaryocytes and/or platelets produced therefrom would not be achievable.

In particular embodiments, the systems and methods utilize a combination of deliberately chosen (1) two or more cell populations at an initial or at least early starting step for co-culture with (2) a particular combination of reagents (including all or at least some of which are cytokines) for the co-culture; the combination of reagents may be provided in the media and are exogenously added to the media. In alternative embodiments, starting cells are manipulated to express one or more of the exogenous reagents, including one or more of SCF, TPO, and IL-6 (which may be referred to as the SCF+TPO+IL-6 cocktail). The SCF+TPO+IL-6 cocktail may comprise any suitable concentration of the three components, which may be 50 ng/ml. Other cytokines like IL-21 (50-150 ng/ml), IL-11 (10-100 ng/ml), IL-9 (40-100 ng/ml), FLT3-L (10-25 ng/ml) individually with SCF+TPO+IL-6 cocktail or in various combinations with the SCF+TPO+IL-6 cocktail can also be applied in specific embodiments for enhanced expansion and differentiation of megakaryocytes. In specific embodiments, the particular combination of reagents comprises, consists essentially or, or consists of SCF, TPO and IL-6. In particular embodiments, the systems and methods also utilize in the combination (3) one or more ROCK inhibitors. In particular embodiments, the systems and methods also (4) have cells in at least one of the two or more cell populations manipulated such that they express one or more exogenous or heterologous genes and/or are manipulated to have knockdown or knock out of one or more endogenous genes in the cells. The exogenous or heterologous gene comprises the HLA class 1 histocompatibility antigen, alpha chain E (HLA-E). In specific cases, cells in at least one of the two or more cell populations are manipulated to be HLA-I depleted and HLA-E overexpressing, which produces megakaryocytes and platelets that are HLA-I depleted and HLA-E overexpressing.

In particular embodiments, the systems and methods utilize media for cell culture as part of the expansion and differentiation and platelet production steps. The media comprises, consists essentially or, or consists of SCF, TPO and IL-6, in specific embodiments. SCF is also known as KIT-ligand, KL, or steel factor, and it is a cytokine that binds to the c-KIT receptor (CD117). SCF can exist both as a transmembrane protein and a soluble protein. In the present systems and methods, any suitable concentration of SCF may be employed, but in specific cases the concentration of SCF is 25-50 ng/ml. In specific cases, the concentration of SCF is 50 ng/mL. TPO is also known as THPO and megakaryocyte growth and development factor (MGDF), and in the present systems and methods, any suitable concentration of TPO may be employed, but in specific cases the concentration of TPO is 50-100 ng/ml. In specific cases, the concentration of TPO is 50 ng/mL. IL-6 is an interleukin that acts as both a pro-inflammatory cytokine, and in the present systems and methods, any suitable concentration of IL-6 may be employed, but in specific cases the concentration of IL-6 is 50-100 ng/ml. In specific cases, the concentration of IL-6 is 50 ng/mL. In specific cases, the concentration of SCF, TPO and IL-6 in the media is the same, whereas in other cases the concentration of SCF, TPO and IL-6 is not the same. In cases wherein the concentration of SCF, TPO and IL-6 is not the same, the concentration level may be determined by the step or steps in the system and methods being considered.

In particular embodiments, the systems and methods utilize a combination of two cell populations as a co-culture that ultimately generates large quantities of functional megakaryocytes. In specific cases, one or both of the two cell populations are derived from a specific source, such as cord blood (CB), bone marrow and/or adipose. The present systems and methods generate large quantities of megakaryocytes from CB hematopoietic progenitors, in at least some cases. In specific embodiments, one of the initial populations of cells includes CD34+ cells, including CD34+ stem cells, such as from CB. In other embodiments, one of the initial populations of cells includes MSCs, including from CB. In one aspect, the system of the disclosure mimics natural processes in the bone marrow microenvironment by having cells in close proximity.

In certain embodiments, the systems and methods produce megakaryocytes and platelets that are HLA-I depleted and HLA-E overexpressing, because they are generated from cells that are HLA-I depleted and HLA-E overexpressing. In specific cases, at least some of the starting and some if not all of the resulting megakaryocytes and platelets have HLA-I knockout and HLA-E knock-in, including HLA-E knock-in at an HLA-I genomic locus in the cells. Such a manipulation greatly reduces or eliminates the risk of transfusion-related graft-versus-host disease or any transfusion refractoriness in recipient individual(s). In specific cases, any suitable HLA-I gene is knocked out, but in specific cases, the HLA-I gene is β2-microglobulin (β2M).

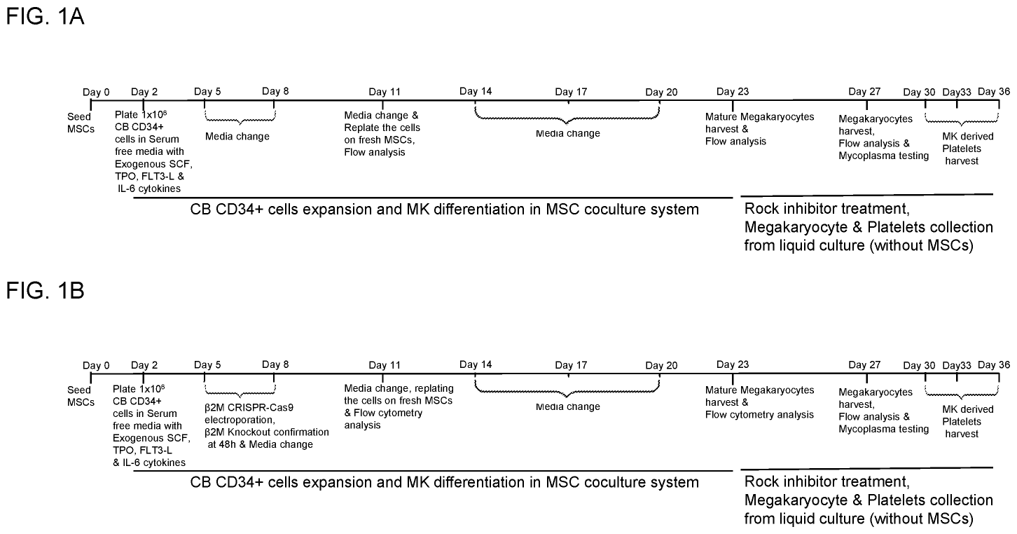

FIG. 1 provides one example of use of the system for megakaryocyte and platelet production. Generally speaking, in specific embodiments, expansion and differentiation occurs to produce mature megakaryocytes, following which the megakaryocytes then produce platelets. Each part of the method (expansion, differentiation, and/or platelet production), or one or more steps therein, may or may not have a specific duration of time, a specific type of media, a specific number of media changes, a specific number of additions of any kind to the media, and so forth. In at least some cases, MSCs are seeded on a substrate or on at least one surface of a vessel. In specific embodiments, the MSCs are adherent to the substrate or surface of the vessel. In at least some cases, a certain percentage of the surface of the substrate or surface of the vessel is covered by adherent MSCs, such as being 40-50% confluent. After a suitable duration of time, such as 1-2 days, CD34+ cells are added to the system. These CD34+ cells may be obtained commercially or may be obtained through the negative depletion of the lineage cell markers, or positive selection of CD34+ cells, or negative/positive selection along with flow cytometry-sorted CD34+ cells prior to use in the system. The CD34+ cells may be stem cells and may be added to the system at a particular range or amount, such as at least 1-5×10⁶ cells. In particular embodiments, the media in the system prior to and/or during the co-culture step is serum-free and comprises, consists of, or consists essentially of reagents that are SCF, TPO, and IL-6.

As illustrated in FIG. 1, the co-culture step with the MSCs and the CD34+ cells is the step in which there is CD34+ expansion and megakaryocyte differentiation. This part of the system may last a number of days, including about 21-30 days. During the expansion and differentiation part of the system and methods, there may or may not be 1, 2, 3, 4, 5, 6, 7, 8, 9, 10, or more changes in media to provide a fresh source of media including reagents of any kind, including SCF, TPO, and IL-6. The concentration of SCF, TPO, and IL-6 during these media changes may or may not be the same concentration of SCF, TPO, and IL-6 as in the first step. In some cases, media changes occur after a specific number of days, such as after 1, 2, 3, 4, 5, or more days, but in specific cases the media is changed after about 3 days. In some cases during the expansion and/or differentiation steps, the cells in culture may or may not be transferred to a different vessel, such as a different flask or bioreactor. In some cases during the expansion and differentiation steps, the cells in culture are re-plated onto fresh MSCs. At any point in time during the methods, including during the expansion and/or differentiation steps, samples from the system may be obtained and analyzed, including for flow cytometry analysis (e.g., to analyze for one or more markers indicative of megakaryocytes) and/or contamination testing. An example of positive markers in the differentiated megakaryocytes includes CD41a, CD42B, CD61, other lineage markers which are negative for CD34 but positive for CD11b, CD14, CD3, CD11c, CD15, CD19, CD56 and CD235a. That is, megakaryocytes progenitors that differentiated originally expressed CD34, but as they became megakaryocytes they lose the CD34 and acquire other markers, in specific embodiments.

As shown in FIG. 1, following a certain number of days in co-culture (e.g., 23-27 days) or following indication of production of megakaryocytes based on testing (including, for example, by flow cytometry and/or other analysis), megakaryocytes are harvested and either stored or further cultured and/or manipulated to produce platelets. In alternative embodiments, the megakaryocytes remain in the system and platelets are harvested from them. In any event, the duration of time from the point of the megakaryocytes being mature enough for platelet production and production of platelets may be 1-10 days, for example. During the platelet collection phase, the system may utilize one or more ROCK inhibitors in the media, such as Y27632, GSK269962, Azaindole 1, RKI-1447, GSK429286a, GSK180736a, fasudil, hydroxyfasudil, or a combination thereof. In some cases, the one or more ROCK inhibitors are added prior to, during, and/or after the megakaryocytes have matured. In some cases, platelets are harvested at multiple times, and the duration between multiple collections may be of any suitable time, such as within 1, 2, 3, 4, 5, or more days of each other. The collected platelets may or may not be combined with other timing of collections, and the platelets may or may not be stored (such as for an off-the-shelf use) prior to use. In some cases, IL-1β cytokine is utilized to facilitate production of platelets and/or TNF-alpha cytokine to increase the platelet aggregation characteristics. In some embodiments, the platelets are transfected or transformed or transduced following collection.

In some cases, steps may be occurring substantially at the same time for different populations of cells in the same system. For example, the system may include an initial expansion of certain cells to produce an expanded population, and at least some of the cells from the expanded population then undergo differentiation, ultimately resulting in production of megakaryocytes. Platelets are then produced from the megakaryocytes. In such a system, however, depending on the timing and conditions, there may be a cell population undergoing expansion in the same system that at substantially the same time also includes a cell population undergoing differentiation.

In particular embodiments, systems and methods of the disclosure produce megakaryocytes and platelets that are genetically modified compared to naturally occurring megakaryocytes and platelets, and such genetic modifications occur because the megakaryocytes and platelets are derived from cells that have been so genetically modified. In some cases, the CD34+ cells and/or MSCs are manipulated such that they express one or more exogenous genes and/or they are manipulated to have knockdown or knock out of one or more endogenous genes in the cells. In specific cases, such as is illustrated in FIG. 1B, the CD34+ cells and/or MSCs are manipulated to comprise a knock-in of HLA class I histocompatibility antigen, alpha chain E (HLA-E) at the locus of one or more HLA-I gene, including at least β2-microglobulin (β2M). The knock-in may be anywhere throughout the locus, including spanning any exon, any intron, or any exon-intron junction. The knock-in may replace all or part of the locus with the HLA-E gene. The knock-in may cause a disruption in the expression of the locus leading to a non-transcribable and/or non-translatable nucleic acid sequence. In some cases, the genetic manipulation of the cells occurs after co-culture has begun, whereas in other cases the genetic manipulation of the cells occurs prior to co-culture. This knock out at PM produces no PM gene product from the locus, in particular aspects. In specific cases, a fusion of HLA-E with PM is not utilized as the construct that is knocked in at PM. Any genetic manipulation of the cells may be by any suitable method, including at least CRISPR, for example.

In the specific example in which HLA-E is knocked in at β2M, the produced platelets are advantageous because they will not elicit or have a reduced capacity to elicit a deleterious immune system reaction upon use in a recipient, compared to platelets produced by cells lacking the HLA-E knock-in at PM. The produced platelets are particularly useful because (1) they have exogenous expression of HLA-E that will provide a signal for native NK cells in the recipient individual not to kill the platelets; and (2) they lack expression of PM that will result in the native T cells in the individual not being able to recognize the transfused platelets, thereby avoiding their destruction by the native T cells. Therefore, the same modification in the platelets (knock-in of HLA-E at the PM locus) allows the platelets to be avoided by both NK cells (by gain of a gene/function) and T cells (by loss of a gene/function).

In some embodiments for the system and methods, the cells in the system are agitated in any manner. In specific cases, the cells are agitated (such as rocked) for a period of time and at a certain part of the method (for example, upon platelet production following production of the megakaryocytes, although in specific cases there is motion of the culture of cells during expansion and/or differentiation). In some cases, the agitation is at a certain angle, such as 8-180 degrees. In at least particular cases, the agitation of the cells at an angle results in shear stress on the megakaryocytes to facilitate platelet release from the megakaryocytes into the media.

In certain embodiments, the media in the system comprises one or more means for fucosylation of megakaryocytes, such as by including one or more fucosyl-transferase enzymes.

At any point in the sequence of events for the method, produced cells and/or cells in production may be suitably stored, such as frozen at a suitable temperature (e.g., −80° C. or in liquid nitrogen). In some cases, the megakaryocytes are stored (such as frozen) prior to production of platelets.

Embodiments of the disclosure encompass systems and methods for producing donor-independent platelets, including for platelet transfusion units, wherein platelets are produced from megakaryocytes that are derived from a co-culture of MSCs and CD34+ cells in the presence of at least TPO, SCF, and IL-6. In at least some cases, any cells during the method are genetically manipulated to be HLA-I depleted and HLA-E overexpressing. In at least some cases, one or more ROCK inhibitors are utilized for any purpose, including for increasing efficacy of platelet production from the megakaryocytes.

### II. Methods of Use of Megakaryocytes and Platelets

Embodiments of the disclosure include methods of using the megakaryocytes and platelets produced by systems and methods of the disclosure. In specific embodiments, an effective amount of platelets (e.g., 1×10⁸ to 1×10¹²) from the produced megakaryocytes are provided to an individual in need thereof. The administration of the platelets to the individual may or may not follow a storage step following the production of the platelets. In specific embodiments, the platelets are HLA-I depleted and HLA-E overexpressing that reduces the chance of deleterious immunoreactivity in the recipient individual. In any event, the individual may be in need of one or more transfusions of platelets, including when the individual is not HLA-matched with a donor. The individual may or may not be receiving platelets as a universal off-the-shelf product, in at least some cases. In any event, the platelets may be transfusion grade.

Any individual in need of platelets may be provide an effective amount of the platelets in any suitable route of administration. In some cases, the individual has cancer; thrombocytopenia for any reason (whether or not with cancer and/or cancer treatment and whether or not from autoimmune or other causes); any bone marrow disease or blood disease that results directly or indirectly in reduced platelet number; anemia; aplastic anemia; coronavirus infection (including SARS-CoV, SARS-CoV-2, MERS, etc.); organ or bone marrow transplants; victim of traumatic injury; individual undergoing heart surgery; burn victim; and so forth. In specific embodiments, a medical facility providing the platelets lacks platelets from an HLA-matched donor. In specific embodiments, the individual is refractory to standard sources of platelets and may have refractory thrombocytopenia.

Methods of treatment with platelets produced by systems and methods of the disclosure include transfusion related graft-versus-host disease or any transfusion refractoriness. In at least specific cases, the individual has a reduced change of transfusion related graft-versus-host disease or any transfusion refractoriness because the platelets are HLA-I depleted and HLA-E overexpressing, allowing both NK cells and T cells to avoid the platelets. Methods and systems of the disclosure circumvent the need for apheresis-derived platelet products for individuals in need thereof for any reason, including at least for individuals undergoing surgery, for an underlying thrombocytopenia for any purpose, receiving chemotherapy, a combination thereof, and so forth.

Embodiments of methods of treating in an individual cancer; thrombocytopenia for any reason (whether or not with cancer and/or cancer treatment and whether or not from autoimmune or other causes); any bone marrow disease or blood disease that results directly or indirectly in reduced platelet number; anemia; aplastic anemia; coronavirus infection (including SARS-CoV, SARS-CoV-2, MERS, etc.); organ or bone marrow transplants; victim of traumatic injury; individual undergoing heart surgery; burn victim; and so forth, comprising the step of providing an effective amount of platelets to the individual, wherein the platelets are produced from systems and methods encompassed herein.

In some embodiments, platelets, including platelet made from megakaryocytes encompassed herein, are lysed to create platelet lysates. The platelet lysates may be used topically. In some embodiments, the platelet lysates are used for hemostasis and/or wound healing. The wounds may be any wound, such as a surgical wound, a diabetic ulcer, or a burn.

### EXAMPLES

The following examples are included to demonstrate particular embodiments of the disclosure. It should be appreciated by those of skill in the art that the techniques disclosed in the examples that follow represent techniques discovered to function well in the practice of the systems and methods of the disclosure, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the systems and methods of the disclosure.

### Example 1

### Production of Megakaryocytes and Platelets

The present example concerns a novel, robust approach for large-scale generation of mature megakaryocytes (MK) from cord blood (CB) using CB tissue-derived allogenic mesenchymal stem cell (MSCs) in a serum free co-culture system with a cocktail of exogenous cytokines (SCF, TPO and IL-6). MSCs from other sources can be used including those from bone marrow and/or adipose tissue, for example. This strategy of ex-vivo expansion and differentiation yields mature megakaryocytes that can efficiently and continuously produce a large number of platelets from the terminally differentiated megakaryocytes (FIG. 1). The platelets can be utilized in platelet transfusion units.

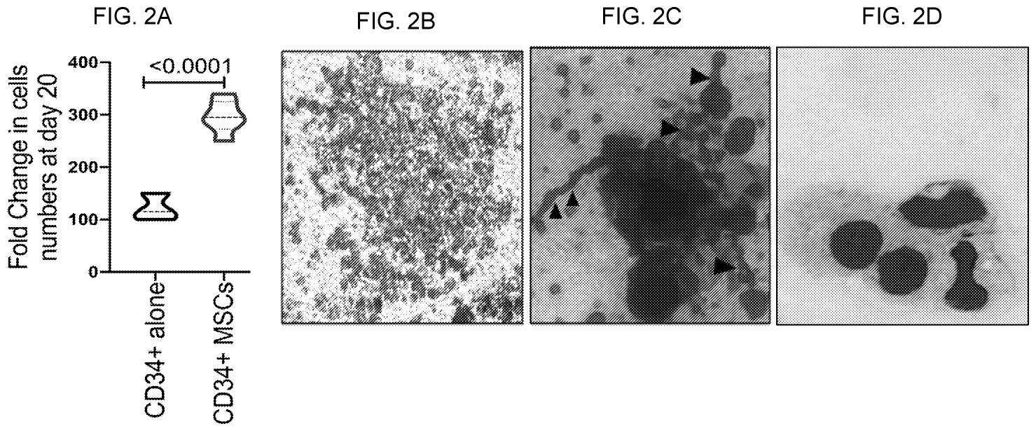

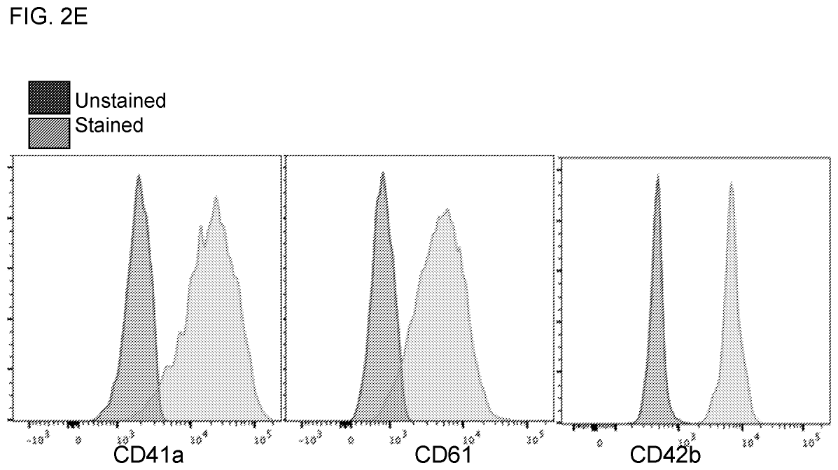

This ex-vivo expansion approach has allowed for the consistent generation of approximately 300-fold expansion over the period of 20 days with the MSCs and generates around 3×10⁸-4×10⁸ megakaryocytes (FIG. 2A). These day 20-expanded mature megakaryocytes can efficiently secrete approximately 1×10¹⁰ functional platelets ex-vivo in each harvest every 3 days. In specific embodiments, with the combination of ROCK inhibitors and additional actions, such as vertical/horizontal shaking to generate shear stress on megakaryocytes, one can generate around 1-5×10¹¹ CB derived platelets. These doses may provide platelet support for individuals in need, such as in thrombocytopenic patients.

The CB derived expanded CD34+ cells have megakaryocyte differentiation potential and can form the megakaryocytes colonies in collagen-based Megacult-C megakaryocyte colony-forming (CFU-Meg) assays, as confirmed by CD41b/CD61+(GPIIb/IIIa receptor complex) staining (FIG. 2B), and were generating platelets in the culture, as evidenced by proplatelets-like structure formation (FIG. 2C). The expanded polyploid megakaryocytes were further verified by Giemsa staining and flow cytometer-based CD42b, CD41a and CD61 expression, all confirming the purity and maturation of the expanded megakaryocytes (FIGS. 2D-2E). These data demonstrate robust differentiation into MK progenitors that have indicators that they have the capacity to provide platelet support.

Rho associated coiled—coil containing protein kinase (ROCK) inhibitors have previously been shown to increase the megakaryocyte cytoskeleton protein remodeling, leading to proplatelet formation and increased platelet shedding from the megakaryocytes [8-10]. The inventors characterized the influence of ROCK inhibitors on CB megakaryocyte ploidy changes, platelets secretions, and their functionality.

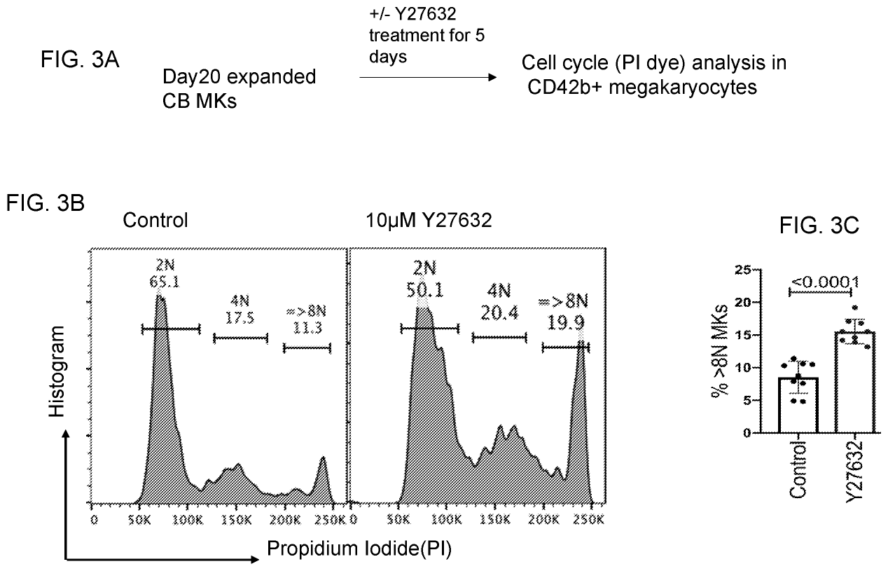

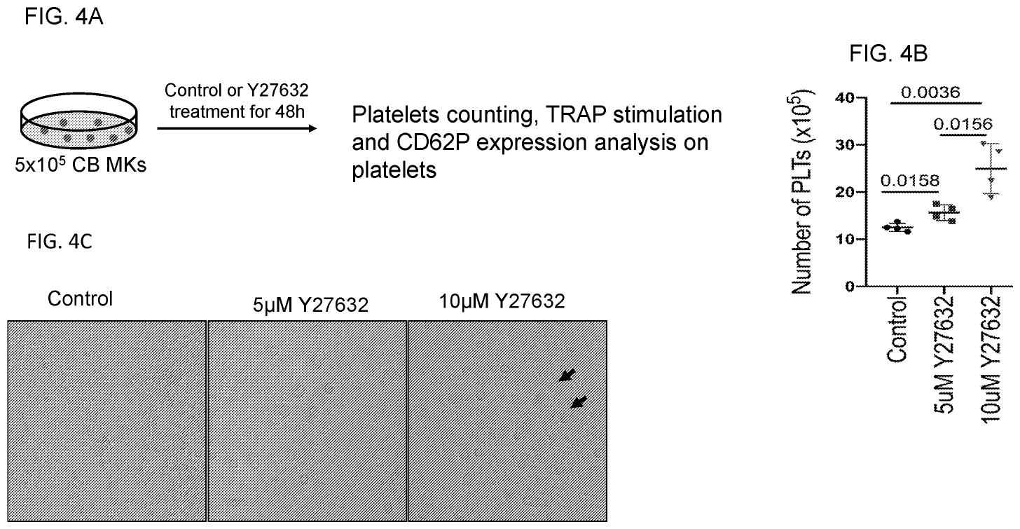

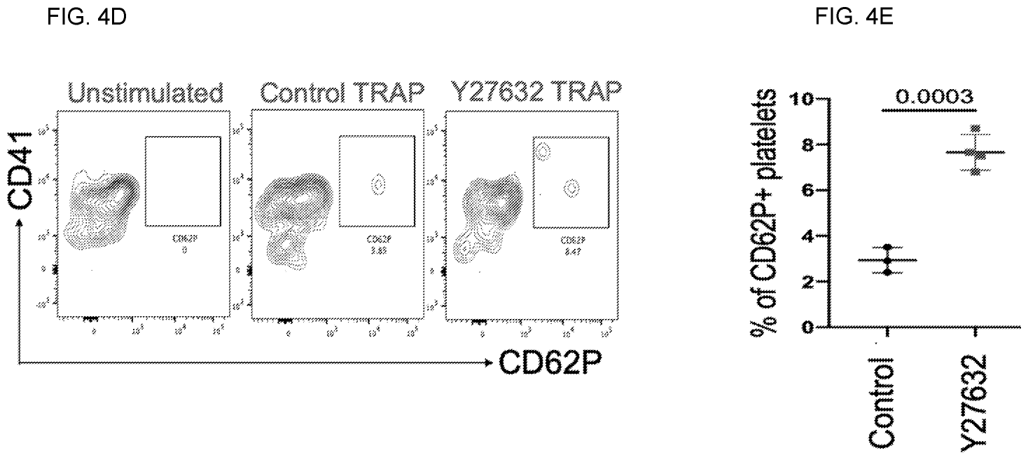

There was a significantly increased number of platelets secreting polyploid megakaryocytes in the ROCK inhibitor-treated group compared to control megakaryocytes, confirming its role in terminal maturation (FIG. 3). Further, the ROCK inhibitor-treated group of MKs secreted a higher number of platelets over 48 hours in a dose-dependent manner (FIGS. 4A-4C) and exhibited enhanced thrombin receptor activating peptides (TRAP)-induced platelet activation and aggregation, as characterized by CD62P expression. This indicates enhancement of functional profile of the CB platelets (FIGS. 4D-4E). Several different ROCK inhibitors can be used in this procedure including Y27632, KD025, GSK269962 and Azaindole 1([9-12].

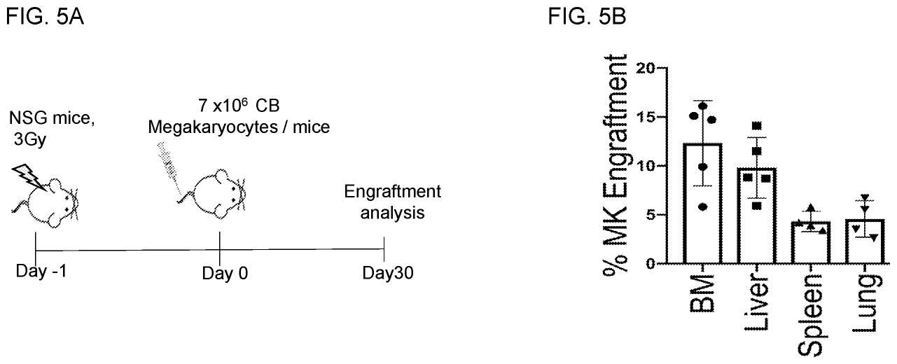

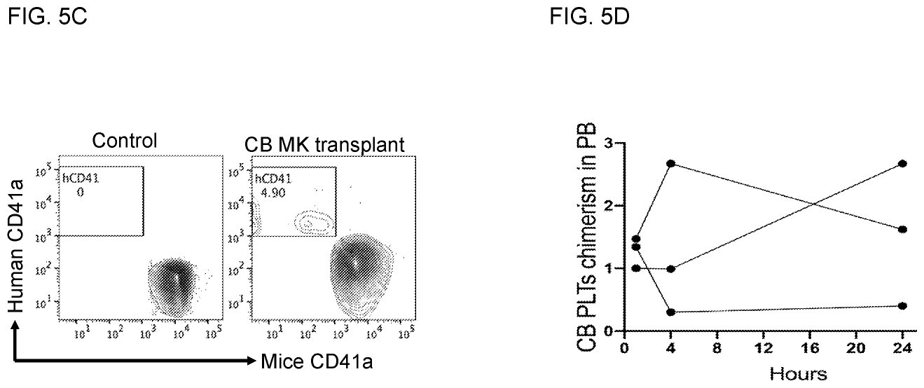

Next, the inventors evaluated the in-vivo functions of the CB-derived expanded megakaryocytes and platelets in a xenograft model for pancyoptenia. First, the thrombocytopenic mice were prepared by sub-lethally (3.5 Gy) radiating the immunodeficient NSG mice, followed by infusion through tail vein route with 7×10⁶ megakaryocytes and 1.3×10⁶ CB platelets/mice in two separate experiments. The ex-vivo generated megakaryocytes and platelets can successfully home and engraft in the immunodeficient mice various organs and secrete circulating platelets in the mice blood (FIGS. 5A-5D).

The use of a ROCK inhibitor in conjunction with the co-culture system represents a novel strategy for faster recovery of platelets counts in individuals receiving CB-derived ex-vivo expanded megakaryocytes and platelets or in chemotherapy patients needing platelet transfusions. In addition to the pharmacological ROCK inhibition, the clustered regularly interspaced short palindromic repeats/Cas9(CRISPR-Cas9) technology can also be applied using ROCK gene(s) specific single guide RNA (sgRNA) or dual guide (crRNA: tracrRNA) RNAs hybrids to knockout the ROCK genes to facilitate the enhanced platelets release from the edited megakaryocytes both in-vitro and in-vivo compared to non-manipulated CB megakaryocytes.

In some embodiments, a bioreactor may be utilized to culture the cells. In a specific case, a GE WAVE bioreactor system is used to culture the ROCK inhibitor-induced terminally differentiated megakaryocyte to liberate platelets. The bioreactor has the capability to culture different cell amounts using various Cellbags™ 2-20 litres for small to scale up of large production for platelets.

Briefly, the CRISPR-Cas9 engineered megakaryocytes are cultured for 24-48 hours in Cellbag™ bioreactor media suspension in 5% CO₂. Hillex® microcarriers are utilized to provide some anchorage support and shear stress. The temperature, cell density, pH levels and viability of the growing megakaryocytes can be continuously monitored. The rocking motion generated with angles in the bioreactor is sufficient enough to induce the shear stress on the surface of megakaryocytes to release the platelets in the media.

After culture, the platelets can be harvested from the Cellbag™ bioreactor suspended media through outlet Clave™ sample port and can be separated further from megakaryocytes using Ficoll-Hypaque-based density gradient centrifugation. The platelets are analyzed for aggregation characteristics using thrombin activating peptides (TRAPs) or ADP stimulation aggregation assays and then used for other downstream applications. The viable megakaryocytes can be reused and (in some cases) cultured with additional megakaryocytes in Cellbag™ to continuously produce the platelets. The IL-1B cytokine can also be used in the suspension media to liberate additional platelets. This approach can be performed with many other bioreactors in clinical use today.

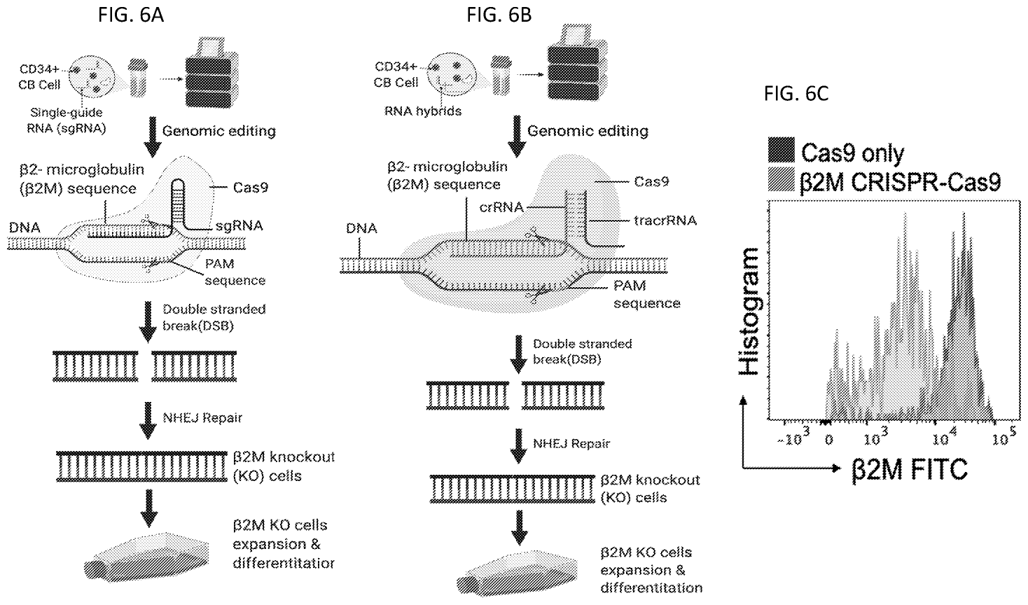

Many cancer patients receiving platelet transfusion develop refractory thrombocytopenia because of the production of alloantibodies against human leucocyte antigen class I (HLA-I). In patients with refractory thrombocytopenia because of HLA antibodies, transfusion of platelets from non-HLA-matched donors fails to increase platelet counts. Hence, in the absence of HLA-I compatible donors, the CB derived platelets transfusions encompassed herein are an effective alternate approach for such patients [13, 14]. HLA-I depleted CB derived platelets are generated using CRISPR-Cas9 technology using single guide RNA (sgRNA, FIG. 6A) or dual guides CRISPR RNA(crRNA) and transactivating crispr RNA (tracrRNA, FIG. 6B) RNAs hybrids to target HLA-I complex molecule β2-microglobulin (β2M) in the CD34+ cells or early megakaryocyte progenitors. The Cas9-induced excision of β2-microglobulin gene exon(s) region produces HLA-1 knock out (KO) or deficient CD34+ cells and CD34+-derived megakaryocytes and platelets. Further, using CRISPR-Cas9/adeno associated vectors (CRISPR-Cas9/AAV) technology to introduce HLA-E gene knock-in (overexpression) and simultaneously removing β2-microglobulin (β2M) gene from the DNA locus in CB CD34+ cells removes at least any minor natural killer (NK) cells associated immune rejection[15].

Further, the ROCK, β2M KO and HLA-E knock-in combination strategy using a novel multiplex CRISPR-Cas9/AAV technology in CB cells is a highly specific tool for genome engineering with high precision. The genome-edited CB megakaryocyte recipients have enhanced platelets secretions and faster platelet counts recovery to alleviate thrombocytopenia while eluding the host immune system rejection, in specific cases.

Apart from CRISPR-Cas9 based genetic engineering manipulation of cell surface expression of HLA genes, the fucosylation of expanded megakaryocytes progenitors or mature megakaryocytes is performed to improve their bone marrow (BM) homing potential, in specific embodiments.

Fucosylation (a type of glycosylation) is carried out by various fucosyl-transferase (FT) enzymes and transfers fucose groups on proteins or carbohydrates moieties, resulting in a de novo acquisition of E-selectin-binding potential. Fucosylated carbohydrate moieties on cell surface are involved in a wide variety of physiological and pathological processes including cell adhesion, leucocytes trafficking and tumor metastasis(16-18). The inventors previously, in a first-in-humans clinical trial, have shown that fucosylation can be increased by ex vivo treatment of CB CD34+ progenitors with short term α1,3-fucosyltransferase (FT-VI) plus GDP-fucose treatment and can significantly reduce the neutropenia and thrombocytopenia duration in the recipient patients (19).

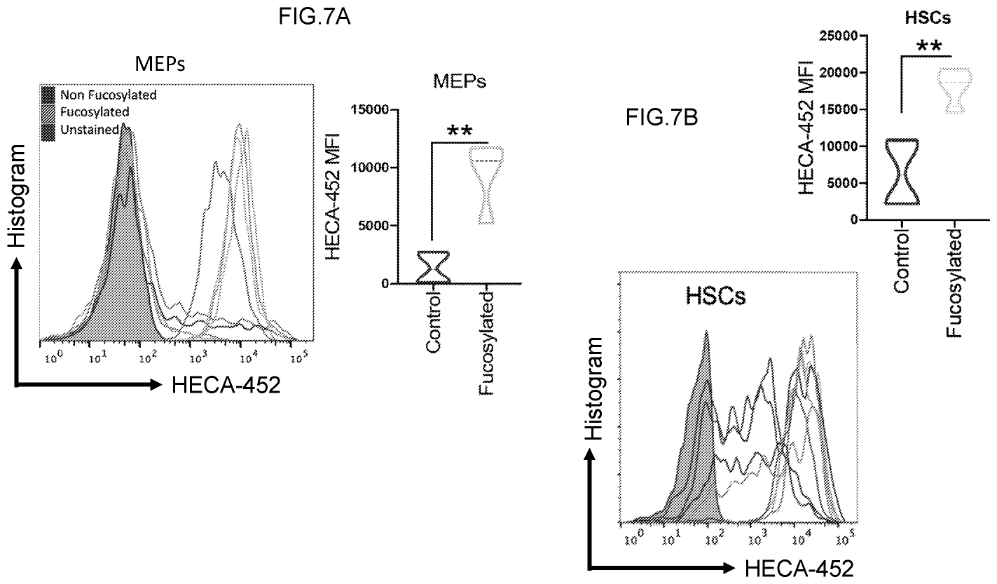

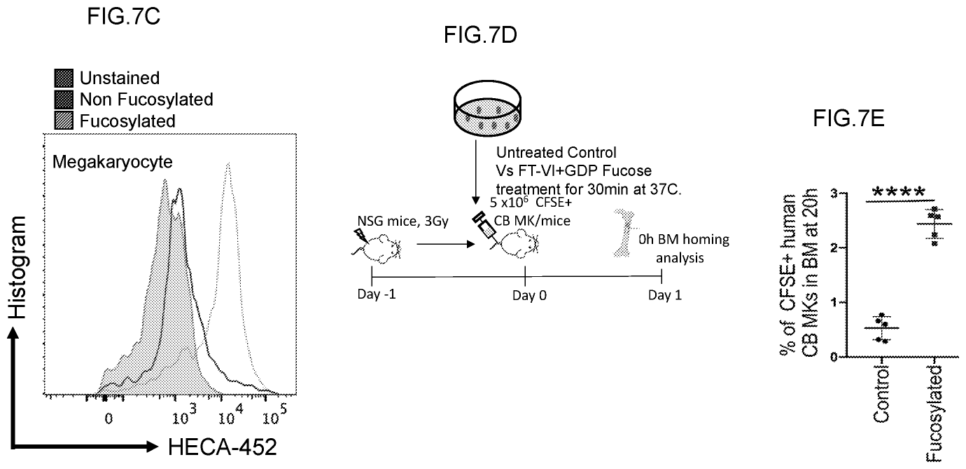

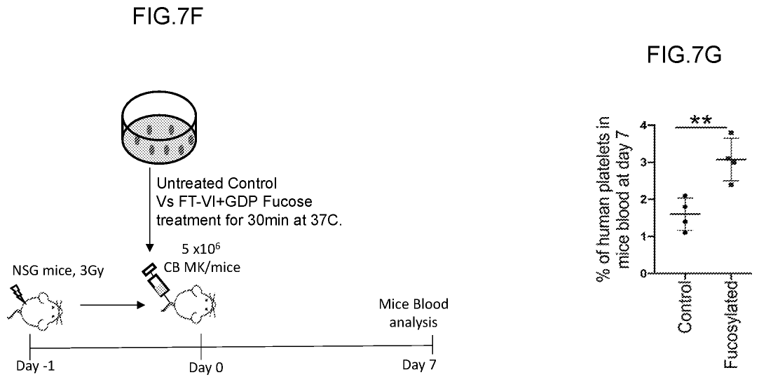

To characterize the fucosylation levels at steady state, the inventors performed flow cytometry HECA-452 antibody staining (which recognizes sLex/cutaneous lymphocyte antigen (CLA), the fucosylated selectin ligand) to measure the levels of cell surface fucosylation in the freshly isolated CB derived hematopoietic stem cells(HSCs, lineage-CD34+CD38-CD90+CD45RA− cells), megakaryocytes lineage committed, megakaryocyte erythroid progenitors (MEPs, lineage-CD34+CD38+CD135−CD45RA−), and differentiated megakaryocytes(CD41a+CD42b+ cells). Further, the ex-vivo treatment with α1,3-fucosyltransferase (FT-VI, 0.025 μg/ml) plus GDP-fucose at 37° C. for 30 min significantly increased the fucosylation levels in HSCs, MEPs and megakaryocytes (FIGS. 7A-7C). The NSG bone marrow homing experiments determine if the CB MK's surface fucosylation modification influences their homing into the irradiated mice marrow niche. Briefly, 5×10⁶ fluorescein succinimidyl ester (CFSE) dye labeled, unmanipulated control or in-vitro fucosyltransferase-VI (FT-VI) induced fucosylated, expanded CB megakaryocytes were injected into sub-lethally (3Gy) irradiated NSG mice and bone marrow homing analysis at 20 h posttransplant was performed by flow cytometry. The percentage homing analysis of CFSE+ megakaryocytes (CD41a+CD42b+) in total live Ter119− (mice RBCs exclusion marker) cells fraction of mouse bone marrow (tibia and femur derived cells) as performed at 20 h and observed a significant higher percentage bone marrow homing in fucosylated megakaryocytes group compared to nonfucosylated megakaryocytes (p<0.0001, FIGS. 7D-7E). Similarly, another megakaryocytes tail vein transplantation (5×10⁶/mice) experiment was performed, and initial percentage circulating platelets levels were measured from the control and fucosylated MK recipient groups. There was a significantly higher level of circulating platelets in fucosylation group mice compared to control MK mice group at day 7 post transplantation (p=0.0058, FIGS. 7F-7G). These data confirmed that increasing the endogenous fucosylation levels will enhance the BM homing of the transplanted megakaryocytes and will boost the megakaryocytes functionality reflected by the circulating platelets. The exogenous FT-VI treatment or overexpression of fucosyltransferase-VI (FT-VI) or fucosyltransferase-VII on CB megakaryocytes (FT-VII) using retroviral, lentiviral based overexpression vectors separately for both genes, both genes combined, and in combination with IL-21 cytokine enhances the BM homing and trafficking in various niches, in specific embodiments. The exogenous fucosylation or endogenous constitutive fucosylation approach with FT-VI or FT-VII enzymes overexpression can be integrated with ROCK inhibition and CRISPR-Cas9 products to generate a unique megakaryocyte product. In at least some cases, these genetically modified MKs and their platelets are useful to be used as an off-the-shelf universal donor product and are an effective strategy to overcome HLA-I-associated alloimmune refractoriness with enhanced BM homing capabilities.

### REFERENCES

All patents and publications cited herein are hereby incorporated by reference in their entirety herein. Full citations for the references cited herein are provided in the following list and are also incorporated by reference herein in their entirety.
1. Freireich E J. Origins of platelet transfusion therapy. Transfus Med Rev. 2011 July; 25(3):252-6.
2. Whitaker B, Rajbhandary S, Kleinman S, Harris A, Kamani N. Trends in United States blood collection and transfusion: results from the 2013 AABB Blood Collection, Utilization, and Patient Blood Management Survey. Transfusion. 2016 September; 56(9):2173-83.
3. Stroncek D F, Rebulla P. Platelet transfusions. Lancet. 2007 Aug. 4; 370(9585):427-38.
4. Stanworth S J, Navarrete C, Estcourt L, Marsh J. Platelet refractoriness—practical approaches and ongoing dilemmas in patient management. Br J Haematol. 2015 November; 171(3):297-305.
Pavenski K, Rebulla P, Duquesnoy R, Saw C L, Slichter S J, Tanael S, et al. Efficacy of HLA-matched platelet transfusions for patients with hypoproliferative thrombocytopenia: a systematic review. Transfusion. 2013 October; 53(10):2230-4.
6. Kerkhoffs J L, Eikenboom J C, van de Watering L M, van Wordragen-Vlaswinkel R J, Wijermans P W, Brand A. The clinical impact of platelet refractoriness: correlation with bleeding and survival. Transfusion. 2008 September; 48(9):1959-65.
7. Saito S, Ota S, Seshimo H, Yamazaki Y, Nomura S, Ito T, et al. Platelet transfusion refractoriness caused by a mismatch in HLA-C antigens. Transfusion. 2002 March; 42(3):302-8.
8. Gobbi G, Mirandola P, Carubbi C, Masselli E, Sykes S M, Ferraro F, et al. Proplatelet generation in the mouse requires PKC epsilon-dependent RhoA inhibition. Blood. 2013 Aug. 15; 122(7):1305-11.
9. Ito Y, Nakamura S, Sugimoto N, Shigemori T, Kato Y, Ohno M, et al. Turbulence Activates Platelet Biogenesis to Enable Clinical Scale Ex Vivo Production. Cell. 2018 Jul. 26; 174(3):636-48 e18.
10. Nakamura S, Takayama N, Hirata S, Seo H, Endo H, Ochi K, et al. Expandable megakaryocyte cell lines enable clinically applicable generation of platelets from human induced pluripotent stem cells. Cell Stem Cell. 2014 Apr. 3; 14(4):535-48.
11. Kumper S, Mardakheh F K, McCarthy A, Yeo M, Stamp G W, Paul A, et al. Rho-associated kinase (ROCK) function is essential for cell cycle progression, senescence and tumorigenesis. Elife. 2016 Jan. 14; 5:e12994.
12. Flynn R, Paz K, Du J, Reichenbach D K, Taylor P A, Panoskaltsis-Mortari A, et al. Targeted Rho-associated kinase 2 inhibition suppresses murine and human chronic GVHD through a Stat3-dependent mechanism. Blood. 2016 Apr. 28; 127(17):2144-54.
13. Gras C, Schulze K, Goudeva L, Guzman C A, Blasczyk R, Figueiredo C. HLA-universal platelet transfusions prevent platelet refractoriness in a mouse model. Hum Gene Ther. 2013 December; 24(12):1018-28.
14. Suzuki D, Flahou C, Yoshikawa N, Stirblyte I, Hayashi Y, Sawaguchi A, et al. iPSC-Derived Platelets Depleted of HLA Class I Are Inert to Anti-HLA Class I and Natural Killer Cell Immunity. Stem Cell Reports. 2020 Jan. 14; 14(1):49-59.
15. Bak R O, Dever D P, Reinisch A, Cruz Hernandez D, Majeti R, Porteus M H. Multiplexed genetic engineering of human hematopoietic stem and progenitor cells using CRISPR/Cas9 and AAV6. Elife. 2017 Sep. 28; 6.
16. Li J, Hsu H C, Mountz J D, Allen J G. Unmasking Fucosylation: from Cell Adhesion to Immune System Regulation and Diseases. Cell Chem Biol. 2018 May 17; 25(5):499-512.
17. Ma B, Simala-Grant J L, Taylor D E. Fucosylation in prokaryotes and eukaryotes. Glycobiology. 2006 December; 16(12):158R-84R.
18. Miyoshi E, Moriwaki K, Nakagawa T. Biological function of fucosylation in cancer biology. J Biochem. 2008 June; 143(6):725-9.
19. Popat U, Mehta R S, Rezvani K, Fox P, Kondo K, Marin D, et al. Enforced fucosylation of cord blood hematopoietic cells accelerates neutrophil and platelet engraftment after transplantation. Blood. 2015 May 7; 125(19):2885-92

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

What is claimed is:

1. A method of producing megakaryocytes in an ex vivo system, comprising the step of co-culturing mesenchymal stem cells (MSCs) with CD34+ cells in one or more vessels or substrates in the presence of media comprising an effective amount of agents, said agents comprising, consisting essentially of, or consisting of stem cell factor (SCF), thrombopoietin (TPO) and interleukin 6 (IL-6), under conditions to produce the megakaryocytes, wherein the CD34+ cells have been manipulated to comprise a knock-in of HLA class I histocompatibility antigen, alpha chain E (HLA-E), at the genomic locus of β2-microglobulin (β2M), thereby reducing or eliminating expression of the HLA class I gene product in the CD34+ cells.

2. The method of claim 1, further comprising the step of enhancing production of platelets from the megakaryocytes.

3. The method of claim 1 or 2, wherein at least the majority of the CD34+ cells and/or the MSCs are derived from cord blood, bone marrow, or adipose tissue.

4. The method of any one of claims 1-3, wherein the vessel further comprises an effective amount of one or more inhibitors of Rho-associated coiled coil containing protein kinase (ROCK).

5. The method of claim 4, wherein the one or more ROCK inhibitors comprises Y27632, GSK269962, Azaindole 1, RKI-1447, GSK429286a, GSK180736a, fasudil, hydroxyfasudil, or a combination thereof.

6. The method of claim 4 or 5, wherein the one or more ROCK inhibitors inhibit ROCK1 and/or ROCK2.

7. The method of any one of the preceding claims, wherein the media lacks serum.

8. The method of any one of the preceding claims, wherein the concentration of SCF is in the range of 25-50 ng/mL.

9. The method of any one of the preceding claims, wherein the concentration of TPO is in the range of 50-100 ng/mL.

10. The method of any one of the preceding claims, wherein the concentration of IL-6 is in the range of 50-100 ng/mL.

11. The method of any one of the preceding claims, wherein the concentration of SCF, TPO, and IL-6 are substantially the same.

12. The method of claim 11, wherein the concentration is 50 ng/mL.

13. The method of any one of the preceding claims, wherein during the co-culturing step and/or during the enhancing platelet production step, the method further comprises agitation of the one or more vessels or substrates.

14. The method of claim 15, wherein the agitation occurs at a desired angle.

15. The method of claim 16, wherein the angle is about 8-9°.

16. The method of any one of claims 13-15, wherein the agitation is sufficient to induce shear stress on the megakaryocytes.

17. The method of any one of claims 2-16, wherein the megakaryocytes are reused to produce additional platelets.

18. The method of any one of the preceding claims, wherein the media comprises an effective amount of IL-1B.

19. The method of any one of claims 1-18, further comprising the step of obtaining a sample of cells from the media to analyze the sample of cells for expression of one or more megakaryocyte markers.

20. The method of claim 19, wherein the sample of cells are obtained from the media about days from the beginning of the co-culture.

21. The method of claim 19, wherein the sample of cells are obtained from the media about 22-24 days from the beginning of the co-culture.

22. The method of any one of claims 19-21, wherein the megakaryocyte markers are selected from the group consisting of CD42b, CD41a, CD61, and a combination thereof.

23. The method of any one of claims 2-22, wherein platelets are obtained from the media.

24. The method of claim 23, wherein platelets are obtained from the media multiple times.

25. The method of claim 24, wherein the duration of time between obtaining the platelets in at least two successive times is about 3 days.

26. The method of any one of claim 2-25, wherein the platelets are analyzed.

27. The method of claim 26, wherein the platelets are analyzed for aggregation.

28. The method of any one of the preceding claims, further comprising the step of subjecting the MSCs, the CD34+ cells, and/or megakaryocytes to an effective amount of one or more means of fucosylation of the CD34+ cells, MSCs, and/or megakaryocytes.

29. The method of claim 28, wherein the means of fucosylation comprises one or more fucosyl-transferase enzymes along with GDP fucose substrate.

30. The method of any of the preceding claims, wherein the media comprises an effective amount of one or more fucosyl-transferase enzymes.

31. A method of producing platelets that avoid a host individual's deleterious immune reaction, comprising the steps of:
(a) co-culturing mesenchymal stem cells (MSCs) with CD34+ cells in one or more vessels or substrates in the presence of media comprising an effective amount of agents, said agents comprising, consisting essentially of, or consisting of SCF, TPO and IL-6, under conditions to produce the megakaryocytes, wherein the CD34+ cells have been manipulated to comprise a knock-in of HLA-E at the genomic locus of B2M, thereby reducing or eliminating expression of B2M in the CD34+ cells, thereby producing megakaryocytes; and
(b) subjecting the megakaryocytes to suitable conditions to produce an effective amount of the platelets.

32. The method of claim 31, wherein the suitable conditions of step (b) comprise an effective amount of one or more ROCK inhibitors in the media.

33. The method of claim 31 or 32, wherein (a) and (b) occur in the same vessel or substrate.

34. The method of any one of claims 31-33, wherein an effective amount of the platelets are provided to the host individual in need thereof.

35. The method of claim 34, wherein the individual in need thereof has cancer; thrombocytopenia; bone marrow disease; blood disease; anemia; aplastic anemia; coronavirus infection; is receiving and/or will receive an organ or bone marrow transplant; has a traumatic injury; is an individual undergoing and/or that will undergo heart surgery; is a burn victim; or a combination thereof.

36. A method of treating an individual in need of platelets, comprising the step of administering to the individual an effective amount of platelets produced by the method of any one of claims 2-30, wherein the individual has cancer; thrombocytopenia; bone marrow disease; blood disease; anemia; aplastic anemia; coronavirus infection; is receiving and/or will receive an organ or bone marrow transplant; has a traumatic injury; is an individual undergoing and/or that will undergo heart surgery; is a burn victim; or a combination thereof.

37. A system comprising, consisting of, or consisting essentially of an effective amount of the following:
MSCs;
CD34+ cells, or, optionally, CD34+ cells comprising a knock in of HLA-E at the B2M genomic locus;
a vessel or substrate;
media;
SCF;
TPO;
IL-6; and, optionally
one or more ROCK inhibitors; and, optionally,
one or more fucosyl-transferase enzymes.
